(19) United States
(12) Patent Application Publication
(10) Pub. No.: US 2026/0345274 A1
(43) Pub. Date: Nov. 5, 2026

(54) SURGICAL ROBOTIC SYSTEM AND METHOD FOR CABLE FATIGUE ESTIMATION OF SURGICAL INSTRUMENTS

(71) Applicant: Covidien LP, Mansfield, MA (US)

(72) Inventors: Stefan Boson Liu, Cambridge, MA (US); Brian A. Rockrohr, Guilford, CT (US)

(21) Appl. No.: 19/488,966

(22) PCT Filed: Jun. 3, 2024
(86) PCT No.: PCT/US2024/032185

§ 371 Date: Dec. 1, 2025

Related U.S. Application Data
(60) Provisional application No. 63/471,854, filed on Jun. 8, 2023.

(51) Int. Cl. A61B 34/00; A61B 34/20; A61B 34/30; A61B 90/00

(52) U.S. Cl. CPC A61B 34/71; A61B 34/30; A61B 2034/2059; A61B 2090/031; A61B 2090/061; A61B 2090/066; A61B 2090/0803; A61B 2090/0809

(57) ABSTRACT

A surgical robotic system includes an instrument drive unit having at least one motor. The system also includes a surgical instrument having at least one pulley, an end effector pivotable about the at least one pulley, and a cable wrapped about the at least one pulley and actuatable by the at least one motor. The system further includes a controller configured to calculate an estimated damage score for the cable, compare the estimated damage score to a threshold, and output an alert in response to the estimated damage score exceeding the threshold.

### CROSS-REFERENCE TO RELATED APPLICATION

The present application claims priority to and benefit of U.S. Provisional Patent Application No. 63/471,854, filed on Jun. 8, 2023, the entire disclosure of which is incorporated by reference herein.

### BACKGROUND

Surgical robotic systems are currently being used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. Surgical instruments may be cable-actuated that are kept under tension to pivot, tilt, and actuate the end effector. In operation, the robotic arm is moved to a position over a patient and then guides the surgical instrument into a small incision via a surgical port or a natural orifice of a patient to position the end effector at a work site within the patient's body. A laparoscopic camera, which is also held by one of the robotic arms, is inserted into the patient to image the surgical site.

Instrument drive cable fatigue is the leading cause of failure for instruments in surgical robotics. Accurately predicting when an instrument will fail is important to ensure safe use of these reusable instruments. Instrument expiration is currently driven by use duration. However, user-behavior can significantly influence how long an instrument lasts before failure. For example, a stationary instrument used to retract tissue will last much longer than a comparable instrument that is subjected to large articulation and actuation motion. Thus, duration alone is an insufficient factor for failure prediction.

### SUMMARY

The present disclosure provides a system and method, which may be implemented as software instructions executable by a processor, for tracking drive cable fatigue. The system and method use a novel and accurate model for cable fatigue prediction, which may be used to estimate instrument expiration based on actual wear and tear that the instrument has sustained. This allows for maximizing the time surgeons can safely use the instruments.

A cable fatigue tracking model according to the present disclosure utilizes position and torque sensors to estimate when an instrument is approaching failure. The model includes factors such as cable tension, bending cycles, bending location, bending radii, and environmental conditions. Given these factors, a damage score is assigned based on a fatigue model using a stress-life curve for different segments of a cable based on the measured instrument usage. Bending locations and cable tensions are estimated by computing the kinematics and dynamics of the instrument.

According to one embodiment of the present disclosure, a surgical robotic system is disclosed. The surgical robotic system includes an instrument drive unit having at least one motor. The system also includes a surgical instrument having at least one pulley, an end effector pivotable about the at least one pulley, and a cable wrapped about the at least one pulley and actuatable by the at least one motor. The system further includes a controller configured to calculate an estimated damage score for the cable, compare the estimated damage score to a threshold, and output an alert in response to the estimated damage score exceeding the threshold.

Implementations of the above embodiment may include one or more of the following features. According to one aspect of the above embodiment, the controller may be further configured to calculate the estimated damage score based on cable tension, a number of bending cycles, and/or a bending location. The instrument drive unit may further include a position sensor configured to measure a rotational position of the at least one motor and a torque sensor configured to measure torque of the at least one motor. The controller may be also configured to calculate displacement of the cable based on the measured rotational position. The controller may be additionally configured to calculate the cable tension, the number of bending cycles, and/or the bending location based on the measured torque and the displacement of the cable using a dynamic model simulating the surgical instrument. The controller may be further configured to store a segmented model of the cable which may include a plurality of segments, and to calculate the number of bending cycles for each segment of the plurality of segments. The controller may be also configured to limit torque output of the at least one motor in response to the estimated damage score exceeding the threshold.

According to another embodiment of the present disclosure, a method for controlling a surgical robotic instrument is disclosed. The method includes actuating a surgical instrument using an instrument drive unit, which includes at least one motor. The surgical instrument includes at least one pulley, an end effector pivotable about the at least one pulley, and a cable wrapped about the at least one pulley and actuatable by the at least one motor. The method also includes calculating, at a controller, an estimated damage score for the cable. The method further includes comparing, at the controller, the estimated damage score to a threshold and outputting on a display an alert in response to the estimated damage score exceeding the threshold.

Implementations of the above embodiment may include one or more of the following features. According to one aspect of the above embodiment, the estimated damage score is calculated based on cable tension, a number of bending cycles, and/or a bending location. The method may also include measuring, at a position sensor, a rotational position of the at least one motor; and measuring, at a torque sensor, a torque of the at least one motor. The method may further include: calculating displacement of the cable based on the measured rotational position. The cable tension, the number of bending cycles, and/or the bending location may be calculated based on the measured torque and the displacement of the cable using a dynamic model simulating the surgical instrument. The method may also include storing a segmented model of the cable which may include a plurality of segments and calculating the number of bending cycles for each segment of the plurality of segments. The method may additionally include limiting torque output of the at least one motor in response to the estimated damage score exceeding the threshold.

According to a further embodiment of the present disclosure, a method for estimating cable fatigue in a cable-driven robotic surgical instrument is disclosed. The method includes actuating a surgical instrument using an instrument drive unit having one or more motors. The surgical instrument includes: one or more pulleys, an end effector pivotable about one or more pulleys, and a cable wrapped around one or more pulleys and actuatable by one or more motors. The method also includes measuring rotational position of one or more motors using a position sensor and torque exerted by one or more motors using a torque sensor. The method additionally includes calculating displacement of the cable based on the rotational position and calculating cable tension based on the toque. The method further includes calculating a number of bending cycles of the cable based on the displacement of the cable and the cable tension and calculating a damage score for the cable based on the number of bending cycles. The method also includes comparing the damage score to a threshold and outputting an alert if the threshold is exceeded.

Implementations of the above embodiment may include one or more of the following features. According to one aspect of the above embodiment, the method may also include generating a segmented model of the cable with a plurality of segments, each representing a portion of the cable and calculating the number of bending cycles for each of the plurality of segments of the cable. The method may further include calculating the damage score for each of the plurality of segments of the cable based on the number of bending cycles. The method may additionally include slowing or stopping one or more motor or limiting the torque output of one or more motors in response to the damage score exceeding the threshold.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a movable cart according to an embodiment of the present disclosure;

FIG. 2 is a perspective view of a surgical robotic arm of the surgical robotic system of FIG. 1 according to an embodiment of the present disclosure;

FIG. 3 is a perspective view of a movable cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of movable carts of FIG. 1 positioned about a surgical table according to an aspect of the present disclosure;

FIG. 6 is a perspective view, with parts separated, of an instrument drive unit and a surgical instrument according to an embodiment of the present disclosure;

FIG. 7 is a top, perspective view of a grasper end effector, according to an embodiment of the present disclosure, for use in the surgical robotic system of FIG. 1;

FIG. 8 is a top, perspective view of a shears end effector, according to an embodiment of the present disclosure, for use in the surgical robotic system of FIG. 1;

FIG. 9 shows the end effector in various configurations according to an embodiment of the present disclosure;

FIG. 10 is a schematic diagram of a system for determining phases of a surgical procedure according to an embodiment of the present disclosure;

FIG. 11 is a schematic diagram of a cable use-life estimation module for tracking drive cable fatigue according to an embodiment of the present disclosure;

FIG. 12 is a schematic diagram of components of the cable use-life module according to an embodiment of the present disclosure;

FIG. 13 is a 3D visualization of a cable fatigue model according to an embodiment of the present disclosure;

FIG. 14 is a schematic diagram of cable segments of the cable fatigue model according to an embodiment of the present disclosure;

FIG. 15 is a stress-life curve for predicting cable fatigue according to an embodiment of the present disclosure; and

FIG. 16 is a flow chart of a method for cable fatigue estimation according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more movable carts having a surgical robotic arm coupled to a setup arm. The surgeon console receives user input through one or more interface devices. The input is processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement commands and to generate a torque commands for activating one or more actuators of the robotic arm, which would, in turn, move the robotic arm in response to the movement commands.

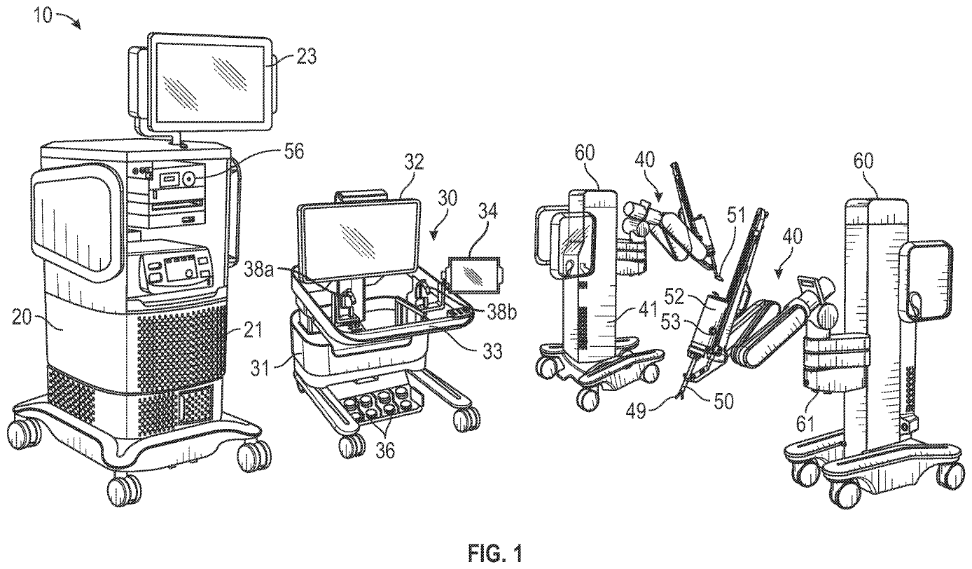

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 coupled thereto. The robotic arms 40 also couple to the movable carts 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured to apply a surgical clip onto tissue. However, it will be understood that various types of surgical instruments for use during minimally invasive surgical procedures are contemplated and within the scope of this disclosure.

One of the robotic arms 40 may include an endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The surgeon console 30 includes a first display 32, which displays a video feed of the surgical site provided by camera 51 disposed on the robotic arm 40, and a second display 34, which displays a user interface for controlling the surgical robotic system 10. The first display 32 and second display 34 may be touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen that may display the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b. The foot pedals 36 may be used to enable and lock the handle controllers 38a and 38b, repositioning camera movement and electrosurgical activation/deactivation. In particular, the foot pedals 36 may be used to perform a clutching action on the handle controllers 38a and 38b. Clutching is initiated by pressing one of the foot pedals 36, which disconnects (i.e., prevents movement inputs) the handle controllers 38a and/or 38b from the robotic arm 40 and corresponding instrument 50 or camera 51 attached thereto. This allows the user to reposition the handle controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted by using any logic processor (e.g., control circuit) adapted to execute algorithms, calculations, and/or set of instructions described herein.

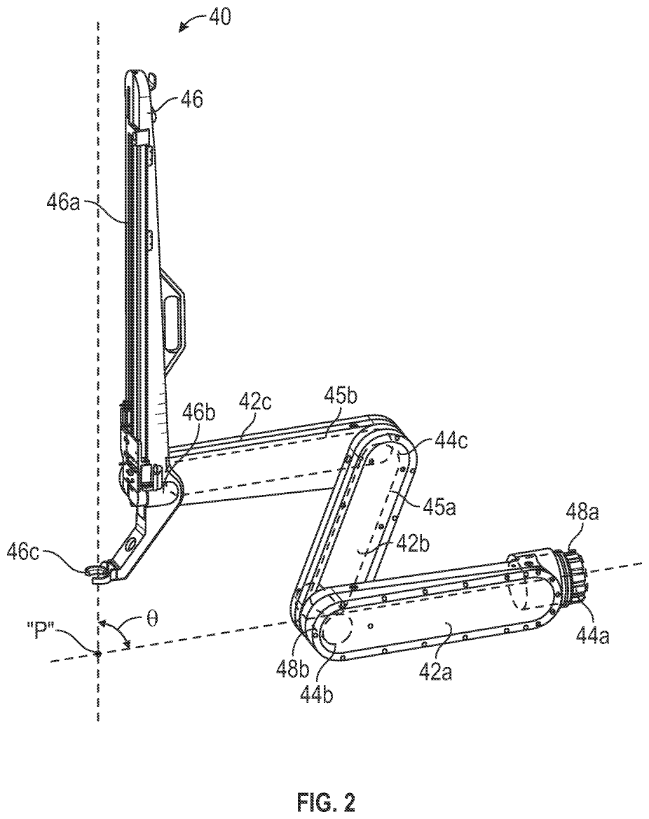

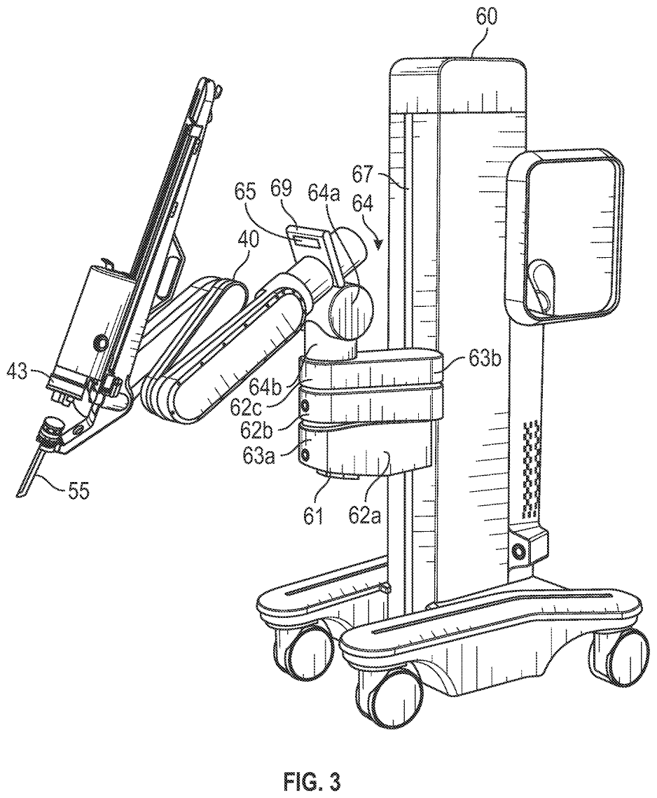

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the movable cart 60 and defines a first longitudinal axis. With reference to FIG. 3, the movable cart 60 includes a lift 67 and a setup arm 61, which provides a base for mounting of the robotic arm 40. The lift 67 allows for vertical movement of the setup arm 61. The movable cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components of an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The IDU 52 is attached to the holder 46, followed by a sterile interface module (SIM) 43 being attached to a distal portion of the IDU 52. The SIM 43 is configured to secure a sterile drape (not shown) to the IDU 52. The instrument 50 is then attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52.

The robotic arm 40 also includes a plurality of manual override buttons 53 (FIG. 1) disposed on the IDU 52 and the setup arm 61, which may be used in a manual mode. The user may press one or more of the buttons 53 to move the component associated with the one or more buttons 53.

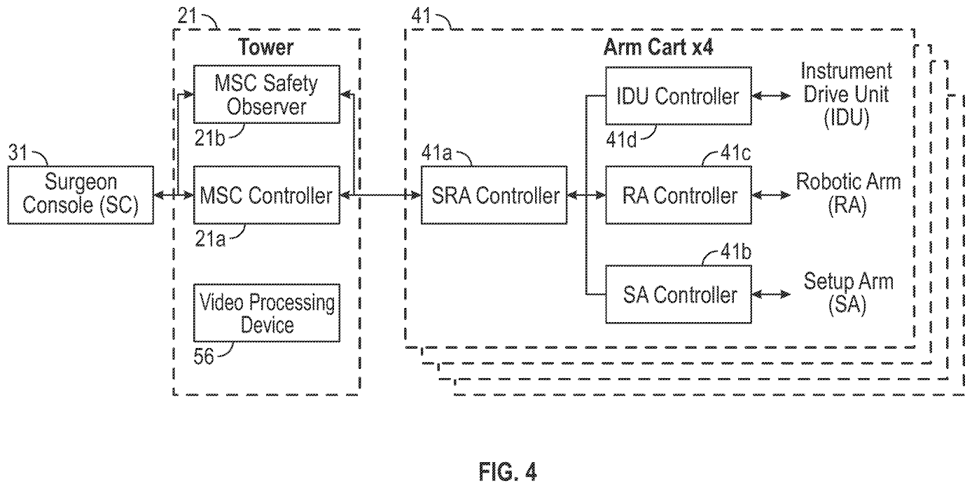

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The desired angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c. In aspects, handle controller 38a may be substituted for and/or employed in conjunction with handle controller 38b. While reference is made above to handle controller 38a, handle controller 38b may also be used in a similar manner.

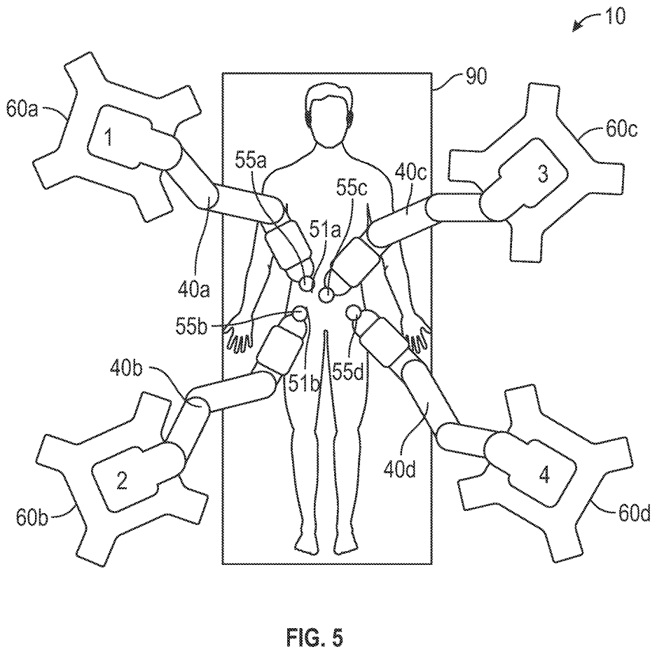

With reference to FIG. 5, the surgical robotic system 10 is setup around a surgical table 90. The system 10 includes movable carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placements are determined, the access ports 55a-d are inserted into the patient, and carts 60a-d are positioned to insert instruments 50 and the endoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46.

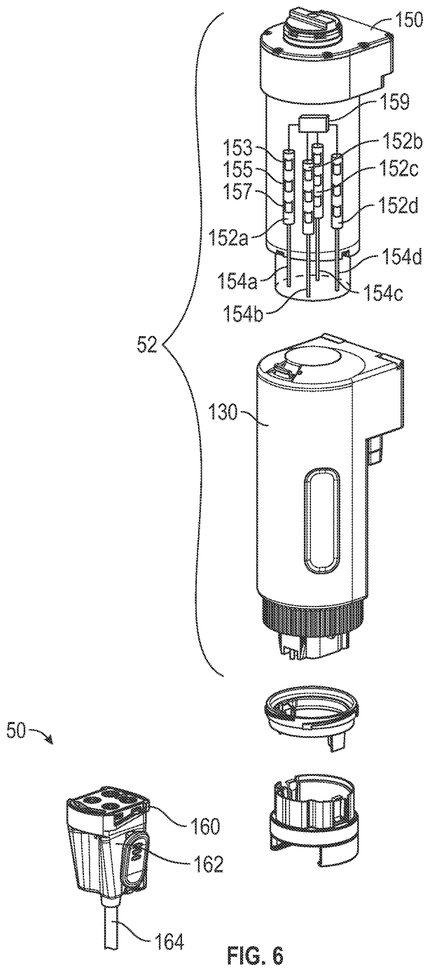

With reference to FIG. 6, the IDU 52 is shown in more detail and is configured to transfer power and actuation forces from its motors 152a, 152b, 152c, 152d to the instrument 50 to drive movement of components of the instrument 50, such as articulation, rotation, pitch, yaw, clamping, cutting, etc. The IDU 52 may also be configured for the activation or firing of an electrosurgical energy-based instrument or the like (e.g., cable drives, pulleys, friction wheels, rack and pinion arrangements, etc.).

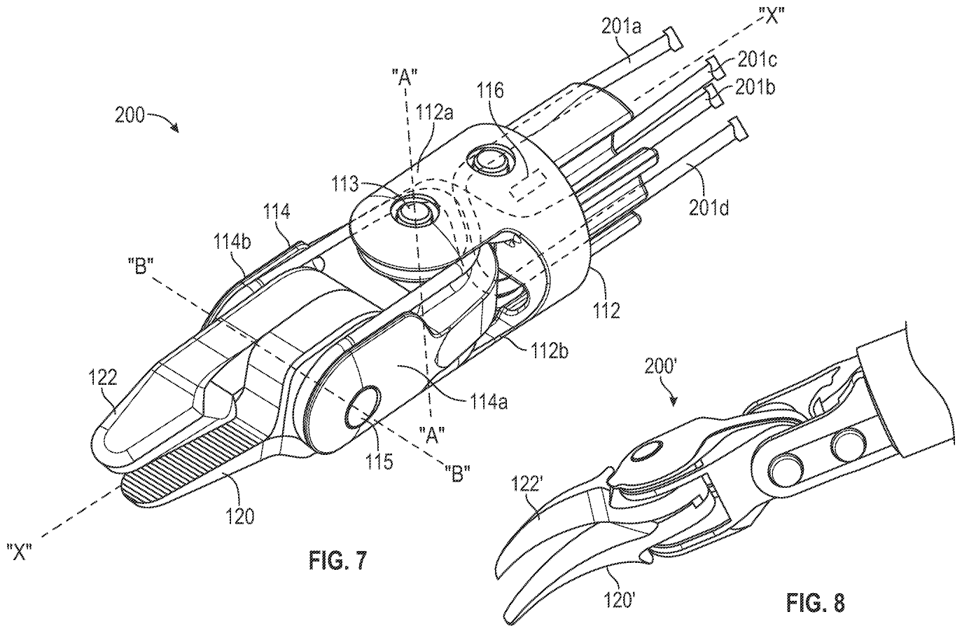

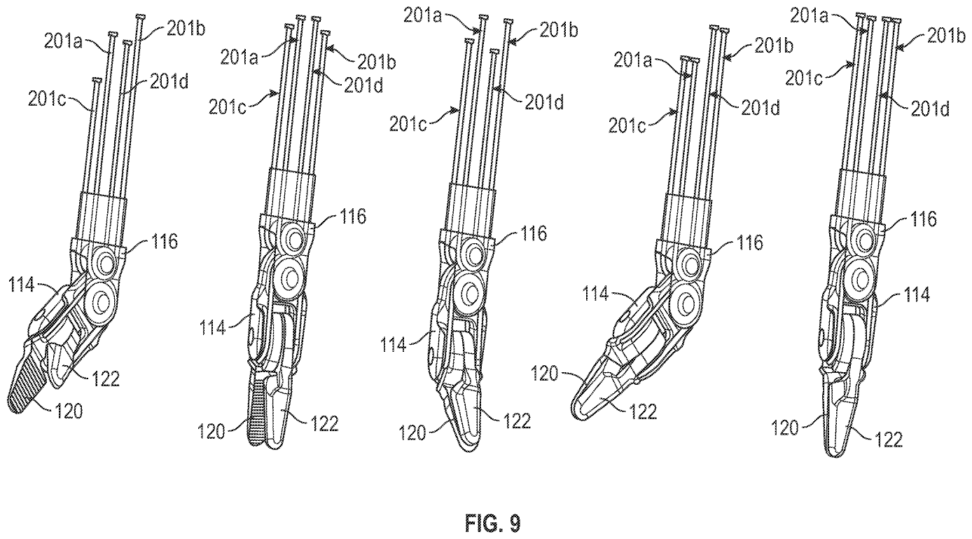

The IDU 52 includes a motor pack 150 and a sterile barrier housing 130. Motor pack 150 includes motors 152a, 152b, 152c, 152d for controlling various operations of the instrument 50. The instrument 50 is removably couplable to IDU 52. As the motors 152a, 152b, 152c, 152d of the motor pack 150 are actuated, rotation of the drive transfer shafts 154a, 154b, 154c, 154d of the motors 152a, 152b, 152c, 152d, respectively, is transferred to the drive assemblies of the instrument 50. The instrument 50 is configured to transfer rotational forces/movement supplied by the IDU 52 (e.g., via the motors 152a, 152b, 152c, 152d of the motor pack 150) into longitudinal movement or translation of the cables or drive shafts to effect various functions of an end effector 200 or 200′ (FIGS. 7 and 8). FIG. 7 shows a grasper end effector 200 and FIG. 8 shows a shears end effector 200′, for simplicity in describing operation of the IDU 52 reference is made only to the end effector 200. The end effector 200′ operates in substantially similar manner to the grasper end effector 200 of FIGS. 7 and 9 but the jaws 120 and 122 are replaced by blade members 120′ and 122′.

Each of the motors 152a, 152b, 152c, 152d includes a current sensor 153, a torque sensor 155, and a position sensor 157. For conciseness only operation of the motor 152a is described below, however, it will be understood that motors 152b-d may operate in a similar manner. The sensors 153, 155, 157 monitor the performance of the motor 152a. The current sensor 153 is configured to measure the current draw of the motor 152a and the torque sensor 155 is configured to measure motor torque. The torque sensor 155 may be any force or strain sensor including one or more strain gauges configured to convert mechanical forces and/or strain into a sensor signal indicative of the torque output by motor 152a. Position sensor 157 may be any device that provides a sensor signal indicative of the number of rotations of the motor 152a, such as a mechanical encoder or an optical encoder. Parameters which are measured and/or determined by position sensor 157 may include speed, distance, revolutions per minute, position, and the like. The sensor signals from sensors 153, 155, 157 are transmitted to the IDU controller 41d, which then controls the motors 152a, 152b, 152c, 152d based on the sensor signals. In particular, the motors 152a, 152b, 152c, 152d are controlled by an actuator controller 159, which controls torque outputted and angular velocity of the motors 152a, 152b, 152c, 152d. In embodiments, additional position sensors may also be used, which include, but are not limited to, potentiometers coupled to movable components and configured to detect travel distances, Hall Effect sensors, accelerometers, and gyroscopes. In embodiments, a single controller can perform the functionality of the IDU controller 41d and the actuator controller 159.

With reference to FIG. 6, instrument 50 includes an adapter 160 having a housing 162 at a proximal end portion thereof and an elongated shaft 164 that extends distally from housing 162. Housing 162 of instrument 50 is configured to selectively couple to IDU 52, to enable motors 152a, 152b, 152c, 152d of IDU 52 to operate the end effector 200 of the instrument 50. Housing 162 of instrument 50 supports a drive assembly that mechanically and/or electrically cooperates with motors 152a, 152b, 152c, 152d of IDU 52. Drive assembly of instrument 50 may include any suitable electrical and/or mechanical component to effectuate driving force/movement.

The surgical instrument also includes an end effector 200 coupled to the elongated shaft 164. The end effector 200 may include any number of degrees of freedom allowing the end effector 200 to articulate, pivot, etc., relative to the elongated shaft 164. The end effector 200 may be any suitable surgical end effector configured to treat tissue, such as a dissector, grasper, sealer, stapler, etc.

As shown in FIGS. 7, 8 and 9, the end effector 200 may include a pair of opposing jaws 120 and 122 that are movable relative to each other. In embodiments, the end effector 200 may include a proximal portion 112 having a first pin 113 and a distal portion 114. Although the jaws 120 and 122 are shown as gripping jaws, it should be understood that the jaws may be any suitable type of jaw, such as shears, etc. The end effector 200 may be actuated using a plurality of cables 201a-d routed through proximal and distal portions 112 and 114 around their respective pulleys 112a, 112b, 114a, 114b, which are integrally formed as arms of the proximal and distal portions 112 and 114. Each of the cables 201a-d is actuated by a respective motor 152a-d via corresponding couplers (not shown) disposed in adapter 160. In embodiments, the end effector 200, namely, the distal portion 114 and the jaws 120 and 122, may be articulated about the axis “A-A” to control a yaw angle of the end effector with respect to a longitudinal axis “X-X”. The distal portion 114 includes a second pin 115 with a pair of jaws including a first jaw 120 and a second jaw 122 pivotably coupled to the second pin 115. The jaws 120 and 122 are configured to pivot about an axis “B-B” defined by the second pin 115 allowing for controlling a pitch angle of the jaws 120 and 122 as well as opening and closing the jaws 120 and 122. The yaw, pitch, and jaw angles between the jaws 120 and 122 as they are moved between open and closed positions are controlled by adjusting the tension and/or length and direction (e.g., proximal or distal) of the cables 201a-d as shown in FIG. 8. The end effector 200 also includes a cable displacement sensor 116 configured to measure position of the cables 201a-d. Thus, the end effector 200 may have three degrees of freedom, yaw, pitch, and jaw angle between jaws 120 and 122.

Wristed end effector 200 utilizes for drive cables 201a-d to articulate pitch, yaw, and jaw degrees of freedom. The cables responsible for closing the jaws are called high-side cables 201b and 201c and those responsible for opening the jaws are called low-side cables 201a and 201d. Thus, the high-side cable 201c and low-side cable 201a actuate the second jaw 122, and high-side cable 201b and the low-side cable 201d actuate the first jaw 120. During closure, the high-side cables 201b and 201c are tensioned while minimum tension is applied to the low-side cables 201a and 201d. During opening, the tension is applied to the cables in reverse, i.e., higher tension to the low-side cables 201a and 201d and minimal tension to the high-side cables 201b and 201c. The cables 201a-d are controlled by their respective motors 152a-d. Thus, the motors 152b and 152c are high-side motors as they actuate high-side cables 201b and 201c and the motors 152a and 152d are low-side motors as they actuate low-side cables 201a and 201d.

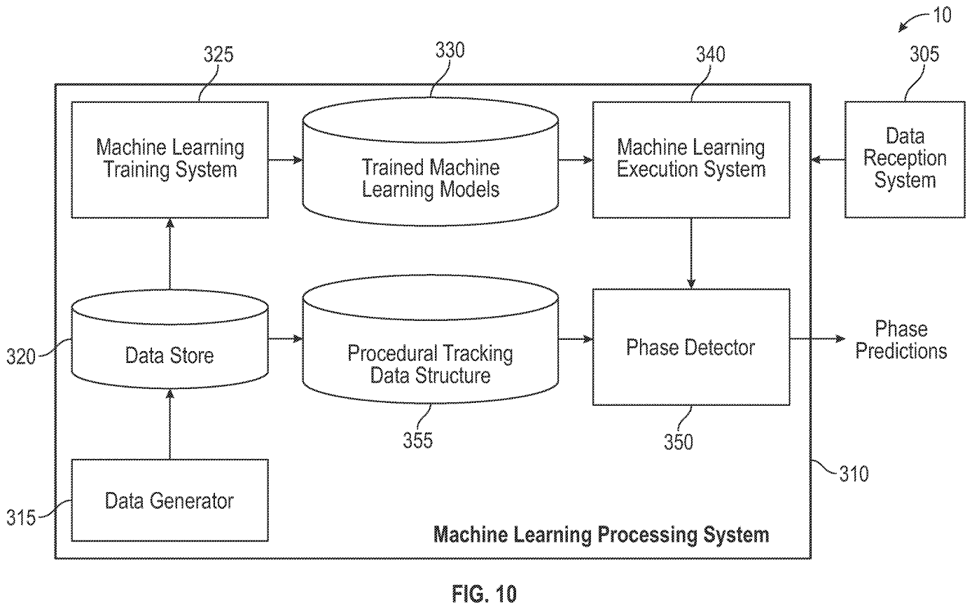

With reference to FIG. 10, the surgical robotic system 10 may include an AI/ML processing system 310 that processes the surgical data using one or more ML models to identify one or more features, such as surgical phase, instrument, anatomical structure, etc., in the surgical data. The ML processing system 310 includes a ML training system 325, which may be a separate device (e.g., server) that stores its output as one or more trained ML models 330. The ML models 330 are accessible by a ML execution system 340. The ML execution system 340 may be separate from the ML training system 325, namely, devices that “train” the models are separate from devices that “infer,” i.e., perform real-time processing of surgical data using the trained ML models 330.

System 10 includes a data reception system 305 that collects surgical data, including the video data and surgical instrumentation data. The data reception system 305 can include one or more devices (e.g., one or more user devices and/or servers) located within and/or associated with a surgical operating room and/or control center. The data reception system 305 can receive surgical data in real-time, i.e., as the surgical procedure is being performed.

The ML processing system 310, in some examples, may further include a data generator 315 to generate simulated surgical data, such as a set of virtual or masked images, or record the video data from the image processing device 56, to train the ML models 330 as well as other sources of data, e.g., user input, arm movement, etc. Data generator 315 can access (read/write) a data store 320 to record data, including multiple images and/or multiple videos.

The ML processing system 310 also includes a phase detector 350 that uses the ML models to identify a phase within the surgical procedure. Phase detector 350 uses a particular procedural tracking data structure 355 from a list of procedural tracking data structures. Phase detector 350 selects the procedural tracking data structure 355 based on the type of surgical procedure that is being performed. In one or more examples, the type of surgical procedure is predetermined or input by user. The procedural tracking data structure 355 identifies a set of potential phases that may correspond to a part of the specific type of surgical procedure.

In some examples, the procedural tracking data structure 355 may be a graph that includes a set of nodes and a set of edges, with each node corresponding to a potential phase. The edges may provide directional connections between nodes that indicate (via the direction) an expected order during which the phases will be encountered throughout an iteration of the surgical procedure. The procedural tracking data structure 355 may include one or more branching nodes that feed to multiple next nodes and/or may include one or more points of divergence and/or convergence between the nodes. In some instances, a phase indicates a procedural action (e.g., surgical action) that is being performed or has been performed and/or indicates a combination of actions that have been performed. In some instances, a phase relates to a biological state of a patient undergoing a surgical procedure. For example, the biological state may indicate a complication (e.g., blood clots, clogged arteries/veins, etc.), pre-condition (e.g., lesions, polyps, etc.). In some examples, the ML models 330 are trained to detect an “abnormal condition,” such as hemorrhaging, arrhythmias, blood vessel abnormality, etc.

The phase detector 350 outputs the phase prediction associated with a portion of the video data that is analyzed by the ML processing system 310. The phase prediction is associated with the portion of the video data by identifying a start time and an end time of the portion of the video that is analyzed by the ML execution system 340. The phase prediction that is output may include an identity of a surgical phase as detected by the phase detector 350 based on the output of the ML execution system 340. Further, the phase prediction, in one or more examples, may include identities of the structures (e.g., instrument, anatomy, etc.) that are identified by the ML execution system 340 in the portion of the video that is analyzed. The phase prediction may also include a confidence score of the prediction. Other examples may include various other types of information in the phase prediction that is output. The predicted phase may be used by the controller 21a to determine when to enable cable fatigue tracking software, e.g., once instrument use is detected.

With reference to FIG. 11, the surgical robotic system 10 may include a cable use-life estimation module 400, which may be implemented as software instructions executable by any one or more of suitable processors of the robotic system 10, such as the IDU controller 41d, the main controller 21a, etc. As used herein, the term module denotes a software component that encapsulates a distinct set of functionality, making it reusable and easily integratable with other parts of the software system. A module typically includes code, data, and resources that work together to perform specific tasks.

The estimation module 400 predicts remaining lifespan of cable-actuated surgical instruments in surgical robotic systems. The estimation module 400 receives as inputs hardware parameters of the surgical instrument 50, including instrument pulley radii and friction, IDU coupler radii, length, and friction, cable properties (e.g., thickness, construction, materials, lubrication, etc.). Additional parameters provided to the estimation module 400 may also include cable tension, number of bending cycles, bending location of the cable, whether the end effector 200 is bent in reverse (which adds additional strain on the cables 201a-d), or whether the instrument 50 is being used in a saline environment (which is corrosive and further decreases cable use-life).

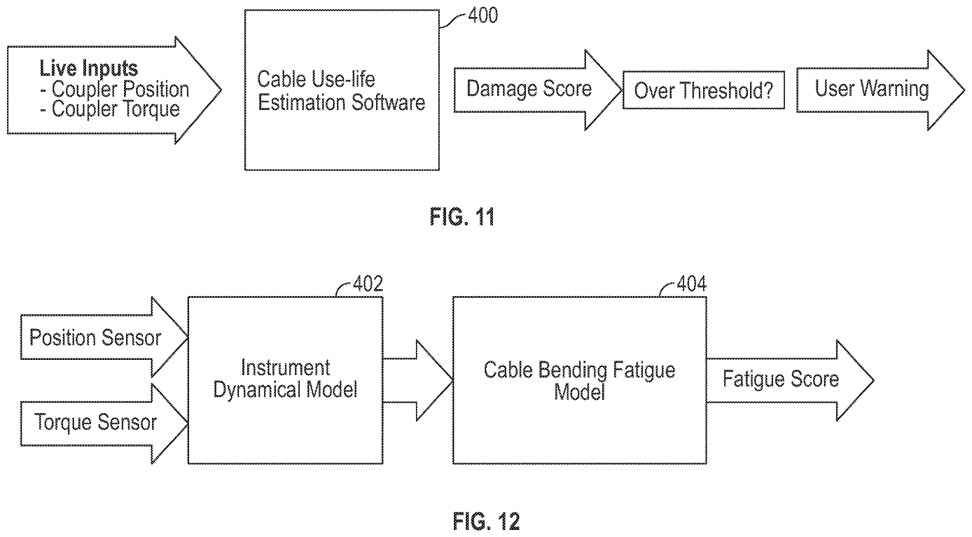

The estimation module 400 also receives sensor feedback from the current sensor 153, the torque sensor 155, and the position sensor 157, which provide current, torque, and position data, respectively, for the motors 152a-d, which actuate the couplers. The estimation module 400 calculates a damage score for each of the cables 201a-d, as described in further detail below with respect to FIGS. 12-15, and compares the damage score to a threshold corresponding to an end of use-life. If the damage score exceeds the threshold, the estimation module 400 then outputs an alert (e.g., auditory or visual) on the surgeon console 30, for example, indicating that the instrument 50 is about to fail, instructing the user to discontinue its use and replace it. In addition, to alerting the surgeon, the estimation module 400 may also take additional remedial action, such scale motion of the motors and/or stop certain motors, requesting that the instrument 50 be withdrawn and replaced.

With reference to FIG. 12, the estimation module 400 includes an instrument dynamic model 402, which simulates the surgical instrument 50 (including its mechanical components and the end effector 200) using the inputs provided to the estimation model 400. The instrument dynamic model 402 functions as a predictive system that simulates and analyzes the behavior of cable-actuated surgical instruments. It utilizes real-time sensor data and predefined parameters to assess bending events and calculate cable tensions, ultimately estimating the remaining use-life of the instrument's cables.

The instrument dynamic model 402 receives input from position and torque sensors to detect bending events in the cables and assess coupler torque and position. The position sensor provides real-time data on the angular or linear displacement of the cable coupler, offering insights into the orientation and position of the end effector 200 during use. Meanwhile, the torque sensor measures the torque applied to the cable coupler, providing information about the forces exerted through the cables.

Hardware parameters such as pulley and coupler radii, distances, and friction coefficients, along with cable properties like material, diameter, and construction, are used to simulate cable behavior. Based on these inputs, the instrument dynamic model 402 detects bending events by analyzing data from position and torque sensors, identifying which cable sections are bent or straight as well as segments that have changed state between straight to bent and vice versa. The instrument dynamic model 402 estimates cable tensions using hardware properties and mathematical calculations to determine the forces acting on each cable segment during bending.

A cable bending fatigue model 404 calculates a cable fatigue score based on output from the instrument dynamic model 402, such as bending events, cable tension, etc. The bending fatigue model 404 uses factors such as tension, bending direction, bending radius, and environmental exposure to determine the cumulative damage on each cable segment. A damage or fatigue score is calculated based on the bending events and stress levels encountered.

The cable bending fatigue model 404 evaluates the cumulative damage that occurs when a cable undergoes bending. The bending fatigue model 404 leverages an exponential-linear relationship between cable tension, bending radius, and the number of bending cycles, incorporating various input parameters that influence cable such as cable tension, bending radius, bending direction, and environmental factors such as exposure to saline conditions or lubrication, and other parameters. These parameters are used to calculate a damage score for each segment of the cable.

The bending fatigue model 404 divides the cable into multiple segments and monitors whether each segment undergoes bending or remains straight during the bending events. This is done because damage accumulates differently at variable stress levels. Dividing the cable into segments and accumulating damage for each bending event allows for tracking damage in a variable manner. Each segment's damage count is updated based on the cumulative effect of bending and reverse bending. When a bending event occurs, the bending fatigue model 404 applies a weighting factor to account for the influence of tension and reverse bending on fatigue accumulation. This results in an updated damage score for each segment based on a linear-exponential tension damage calculation that relates the cable tension and bending radius to the damage accumulated. Damage accumulates differently at variable stress levels, therefore dividing the cable into each segment with individual damage score allows for accurately tracking and/or predicting stress for the cable as a whole of specific segments. The damage score threshold may be empirically derived by experimentation on the cables, by subjecting the cables to multiple bending events, in different environments, and varying other parameters described above.

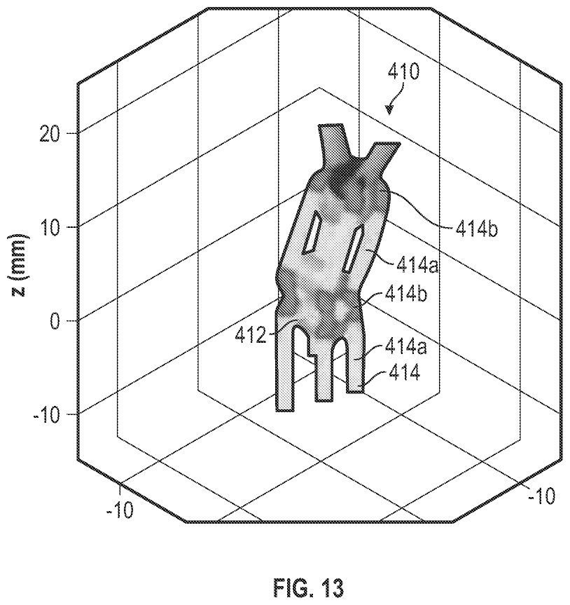

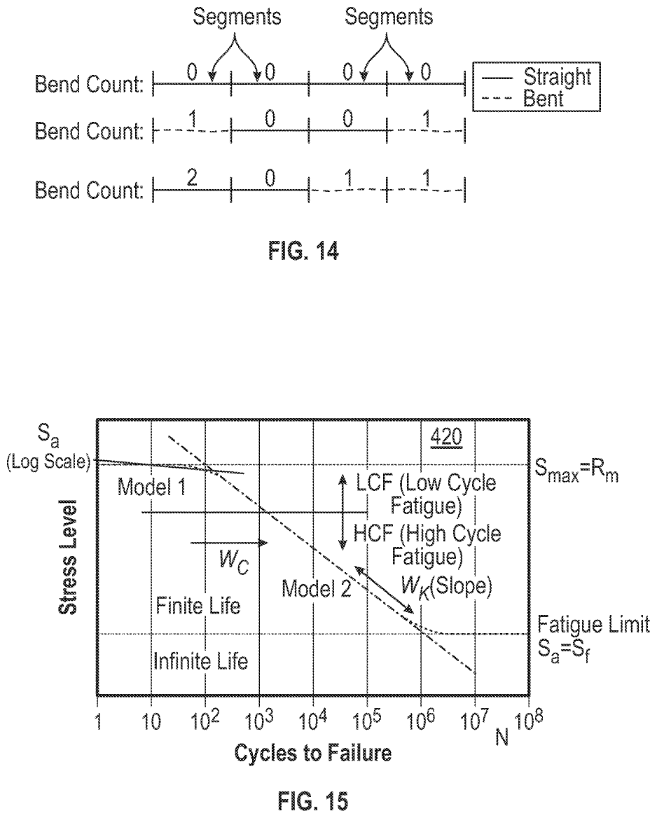

FIG. 13 shows a 3D model 410 generated by the instrument dynamic model 402. The 3D model 410 includes one or more pulleys 412 and cables 414 corresponding to the pulleys 112a, 112b, 114a, 114b and cables 210a-d, respectively. The cables 414 includes a plurality of segments some of which are straight segments 414a and others are bent segments 414b. The bending fatigue model 404 uses instrument kinematics and output of the instrument dynamic model 402 to determine which segments of the cables 414 are bent and which sections are straight. In addition, as the cables 414 are moved longitudinally by the motors 152a-d, the segments can also transition between straight and bent configurations. The tracking of the number of bend counts and current straight or bent configuration is shown in FIG. 14.

FIG. 15 shows a curve 420 illustrating the relation between cable tension and cycle life. The curve 420 is an S-N curve, also known as a Wöhler curve or stress-life curve and is a graphical representation of the relationship between cyclic stress(S) and the number of cycles to failure (N) for a given material. It is used in fatigue analysis to characterize how a material behaves under repetitive loading. The curve 420 shows how many cycles a material, in this case the cable, can withstand at a specific stress level before it ultimately fails. The added stress for each bending even corresponds to the stress of the curve 420.

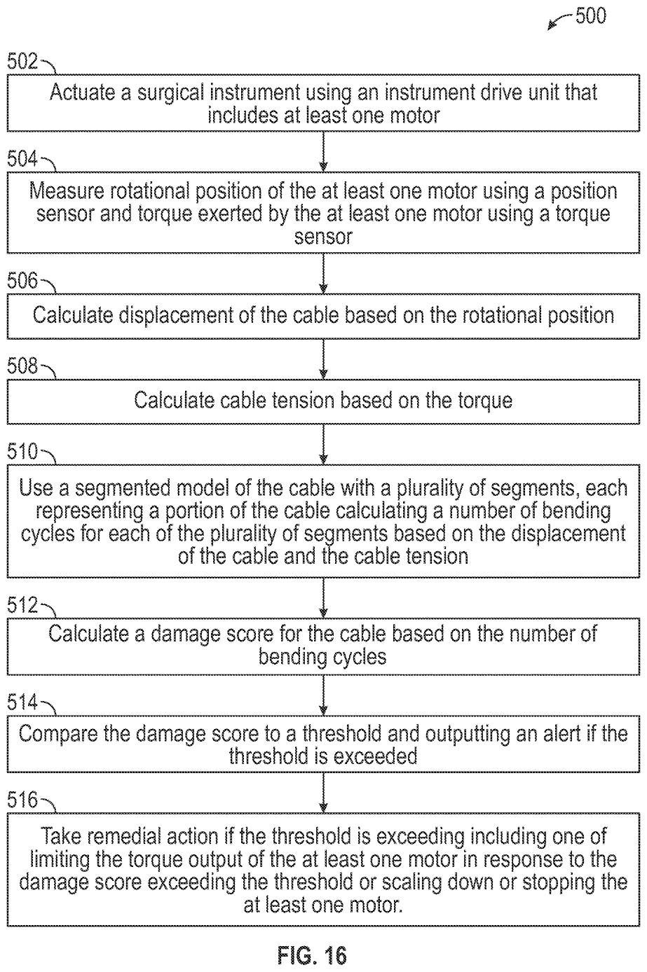

With reference to FIG. 16, an illustrative method 500 for cable fatigue estimation is implemented as software instructions executable by any one or more of suitable processors of the robotic system 10, such as the IDU controller 41d, the main controller 21a, etc. The method 500 includes at step 502 actuating the surgical instrument 50 using the IDU 52. At step 504, motor position, torque, and/or current are measured. These parameters are provided to the cable use-life estimation module 400. The cable use-life estimation module 400 loads or accesses a segmented model of the cable with a plurality of segments and uses the model to monitor specific sections of the cable. At step 506, cable displacement is calculated based on the rotational position data collected. At step 508, the instrument dynamic model 402 is used to calculate the cable tension, the number of bending cycles, and bending location using a dynamic model of the surgical instrument 50. At step 510, the number of bending cycles for each segment of the cable is calculated to determine fatigue accumulation at specific sections. At step 512, a damage score is calculated for the cable based on cable tension, the number of bending cycles, and/or the bending location for each of the segments. At step 514, the calculated damage score is compared to a predetermined threshold. At step 516, the controller 21a may perform one or more alerting actions in response to the damage score exceeding the threshold, which is indicative of a predicted failure of the cable. The controller 21a may output an auditory or visual alert on the surgeon console 30 and/or request replacement of the instrument if the damage score is too high. Additionally, at step 516 the controller 21a may perform one or more remedial actions, such as scale down the motion of the motors controlling the cable at issue or stop specific motors to reduce strain on the cable. The controller 21a may also limit the torque output of the motor in response to the estimated damage score exceeding the threshold, preventing further damage.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
an instrument drive unit including at least one motor;
a surgical instrument including:
at least one pulley;
an end effector pivotable about the at least one pulley; and
a cable wrapped about the at least one pulley and actuatable by the at least one motor; and
a controller configured to:
calculate an estimated damage score for the cable;
compare the estimated damage score to a threshold; and
output an alert in response to the estimated damage score exceeding the threshold.

2. The surgical robotic system according to claim 1, wherein the controller is further configured to calculate the estimated damage score based on at least one of a cable tension, a number of bending cycles, or a bending location.

3. The surgical robotic system according to claim 2, wherein the instrument drive unit further includes:
a position sensor configured to measure a rotational position of the at least one motor; and
a torque sensor configured to measure a torque of the at least one motor.

4. The surgical robotic system according to claim 3, wherein the controller is further configured to calculate displacement of the cable based on the measured rotational position.

5. The surgical robotic system according to claim 4, wherein the controller is further configured to calculate at least one of a cable tension, a number of bending cycles, or a bending location based on the measured torque and the displacement of the cable using a dynamic model simulating the surgical instrument.

6. The surgical robotic system according to claim 5, wherein the cable includes a plurality of segments, and the controller is further configured to store a segmented model of the cable.

7. The surgical robotic system according to claim 6, wherein the controller is further configured to calculate the number of bending cycles for each segment of the plurality of segments.

8. The surgical robotic system according to claim 1, wherein the controller is further configured to limit torque output of the at least one motor in response to the estimated damage score exceeding the threshold.

9. A method for controlling a surgical robotic instrument, the method comprising:
actuating a surgical instrument using an instrument drive unit including at least one motor, the surgical instrument including:
at least one pulley;
an end effector pivotable about the at least one pulley; and
a cable wrapped about the at least one pulley and actuatable by the at least one motor;
calculating, at a controller, an estimated damage score for the cable;
comparing, at the controller, the estimated damage score to a threshold; and
outputting on a display an alert in response to the estimated damage score exceeding the threshold.

10. The method according to claim 9, wherein the estimated damage score is calculated based on at least one of a cable tension, a number of bending cycles, or a bending location.

11. The method according to claim 10, further comprising:
measuring, at a position sensor, a rotational position of the at least one motor; and
measuring, at a torque sensor, a torque of the at least one motor.

12. The method according to claim 11, further comprising:
calculating displacement of the cable based on the measured rotational position.

13. The method according to claim 12, wherein at least one of a cable tension, a number of bending cycles, or a bending location are calculated based on the measured torque and the displacement of the cable using a dynamic model simulating the surgical instrument.

14. The method according to claim 13, wherein the cable includes a plurality of segments, and the method further comprises:
storing a segmented model of the cable.

15. The method according to claim 14, further comprising:
calculating the number of bending cycles for each segment of the plurality of segments.

16. The method according to claim 9, further comprising:
limiting torque output of the at least one motor in response to the estimated damage score exceeding the threshold.

17. A method for estimating cable fatigue in a cable-driven robotic surgical instrument, the method comprising:
actuating a surgical instrument using an instrument drive unit that includes at least one motor, the surgical instrument including:
at least one pulley,
an end effector pivotable about the at least one pulley, and
a cable wrapped around the pulley and actuatable by the at least one motor;
measuring a rotational position of the at least one motor using a position sensor and a torque exerted by the at least one motor using a torque sensor,
calculating displacement of the cable based on the measured rotational position;
calculating cable tension based on the measured toque;
calculating a number of bending cycles of the cable based on the displacement of the cable and the cable tension;
calculating a damage score for the cable based on the number of bending cycles; and
comparing the damage score to a threshold and outputting an alert if the threshold is exceeded.

18. The method according to claim 17, further comprising:
generating a segmented model of the cable with a plurality of segments, each representing a portion of the cable; and
calculating the number of bending cycles for each of the plurality of segments of the cable.

19. The method according to claim 18, further comprising:
calculating the damage score for each of the plurality of segments of the cable based on the number of bending cycles.

20. The method according to claim 17, further comprising:
slowing or stopping the at least one motor or limiting the torque output of the at least one motor in response to the damage score exceeding the threshold.
